(19) United States
(12) Patent Application Publication
(10) Pub. No.: US 2026/0077212 A1
(43) Pub. Date: Mar. 19, 2026

(54) OPTICAL FIBER RETAINER, PHOTOTHERAPY DEVICE, AND PHOTOTHERAPY SYSTEM

(71) Applicant: OLYMPUS CORPORATION, Tokyo (JP)

(72) Inventor: Takumi HAYASHI, Gunma (JP)

(73) Assignee: OLYMPUS CORPORATION, Tokyo (JP)

(21) Appl. No.: 19/337,000

(22) Filed: Sep. 23, 2025

Related U.S. Application Data
(63) Continuation of application No. PCT/JP2023/013480, filed on Mar. 31, 2023.

(51) Int. Cl. A61N 5/067; A61B 1/018; A61N 5/06

(52) U.S. Cl. CPC A61N 5/067; A61B 1/018; A61N 2005/063; A61N 2005/0632; A61N 2005/0659

(57) ABSTRACT

An optical fiber retainer is attached to a side surface of an optical fiber and maintains, within a channel of a medical tube, a fixed distance between the side surface of the optical fiber and an inner surface of the channel. The optical fiber retainer includes a substantially tubular main unit having a fiber hole through which the optical fiber extends. The main unit has a flow-path defining section that defines a flow path within the channel. The flow path allows a fluid to travel in a longitudinal direction of the main unit.

### CROSS-REFERENCE TO RELATED APPLICATIONS

This is a continuation of International Application PCT/JP2023/0013480 which is hereby incorporated by reference herein in its entirety.

### TECHNICAL FIELD

The present invention relates to optical fiber retainers, phototherapy devices, and phototherapy systems.

### BACKGROUND ART

Known lithotripsy in the related art involves using laser light to fragment a calculus occurring in, for example, a kidney (e.g., see Non Patent Literature 1). In order to efficiently fragment the calculus, it is preferable to irradiate the calculus with the laser light while scanning the laser light. Non Patent Literature 1 discloses using a magnet bead fixed to an optical fiber and a solenoid in the vicinity of the optical fiber to vibrate the distal end of the optical fiber, thereby scanning the laser light.

### CITATION LIST

### Non Patent Literature

### {NPL 1}

Layton A. Hall, two others, “Thulium fiber laser stone dusting using an automated, vibrating optical fiber”, Proceedings Volume 10852, Therapeutics and Diagnostics in Urology 2019, Feb. 26, 2019

### SUMMARY OF INVENTION

An aspect of the present invention provides an optical fiber retainer that is attached to a side surface of an optical fiber and that maintains, within a channel of a medical tube, a fixed distance between the side surface of the optical fiber and an inner surface of the channel. The optical fiber retainer includes a substantially tubular main unit having a fiber hole through which the optical fiber extends. The main unit has a flow-path defining section that defines a flow path within the channel. The flow path allows a fluid to travel in a longitudinal direction of the main unit.

Another aspect of the present invention provides a phototherapy device including: a medical tube having a channel; an optical fiber inserted in the channel; and an optical fiber retainer that is disposed between the optical fiber and an inner surface of the channel and that maintains a fixed distance between a side surface of the optical fiber and the inner surface of the channel. The optical fiber retainer includes a substantially tubular main unit having a fiber hole through which the optical fiber extends. The main unit has a flow-path defining section that defines a flow path within the channel. The flow path allows a fluid to travel in a longitudinal direction of the main unit.

Another aspect of the present invention provides a phototherapy system including: a medical tube having a channel; an optical fiber inserted in the channel; an optical fiber retainer that is disposed between the optical fiber and an inner surface of the channel and that maintains a fixed distance between a side surface of the optical fiber and the inner surface of the channel; a laser light source that supplies laser light to the optical fiber; and a fluid supply source that supplies a fluid to the channel. The optical fiber retainer includes a substantially tubular main unit having a fiber hole through which the optical fiber extends. The main unit has a flow-path defining section that defines a flow path within the channel. The flow path allows the fluid to travel in a longitudinal direction of the main unit.

### BRIEF DESCRIPTION OF DRAWINGS

FIG. 1 illustrates the overall configuration of a phototherapy system according to an embodiment of the present invention.

FIG. 2A is a side view of an optical fiber retainer according to the embodiment.

FIG. 2B is a side view of an optical fiber retainer according to another embodiment.

FIG. 3A is a vertical end view of a main unit of the optical fiber retainer, taken along a central axis.

FIG. 3B is a vertical end view of the main unit into which an optical fiber is inserted, taken along the central axis.

FIG. 3C is a cross-sectional view of the main unit, taken in a direction orthogonal to the central axis.

FIG. 4 is a vertical end view of another example of the main unit of the optical fiber retainer, taken along the central axis.

FIG. 5 is a flowchart of a phototherapy method according to an embodiment of the present invention.

FIG. 6A is a vertical end view of another example of the main unit of the optical fiber retainer.

FIG. 6B is a front view of the main unit in FIG. 6A, as viewed from the distal end.

FIG. 7 is a vertical end view of another example of the main unit of the optical fiber retainer.

FIG. 8 is a front view of another example of the main unit of the optical fiber retainer.

### DESCRIPTION OF EMBODIMENTS

An optical fiber retainer, a phototherapy device, and a phototherapy system according to an embodiment of the present invention will be described below with reference to the drawings.

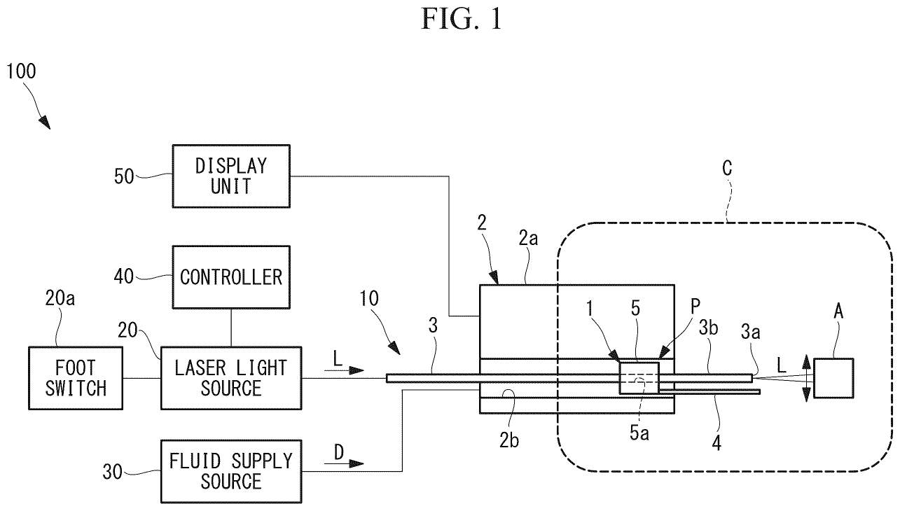

As shown in FIG. 1, a phototherapy system 100 according to this embodiment is a lithotripsy system that uses laser light L to fragment a calculus serving as a treatment target A. The phototherapy system 100 includes a phototherapy device 10, a laser light source 20, a fluid supply source 30, and a controller 40.

The phototherapy device 10 includes a medical tube 2, an optical fiber 3, an optical fiber retainer 1, and an operation member 4.

The medical tube 2 is an endoscope having a long and flexible insertion section 2a. An image inside a body C acquired by the endoscope 2 may be displayed on a display unit 50. The endoscope 2 has a channel 2b extending through the insertion section 2a in the longitudinal direction. The channel 2b is for a perfusate D and the optical fiber 3.

The optical fiber 3 has a distal end 3a that outputs the laser light L and a proximal end that is connected to the laser light source 20. The optical fiber 3 has an outer diameter smaller than the inner diameter of the channel 2b and is insertable into the channel 2b.

The optical fiber retainer 1 includes a substantially cylindrical main unit 5 that retains the optical fiber 3.

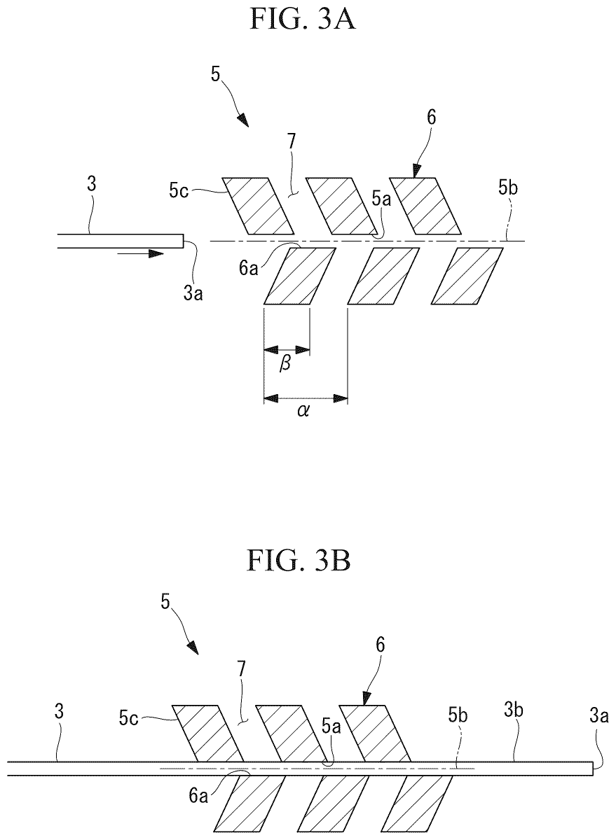

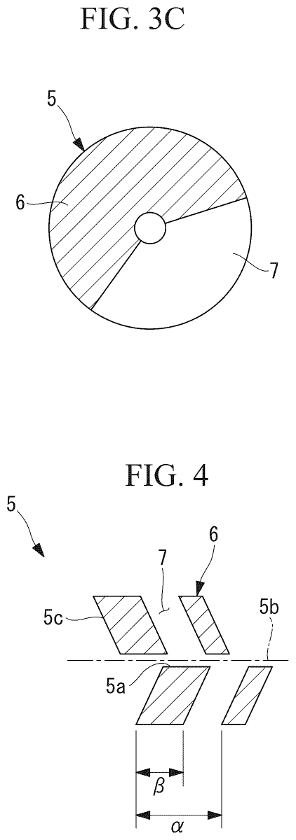

The main unit 5 has a fiber hole 5a extending through the main unit 5 in the longitudinal direction from a distal end surface to a proximal end surface, and is attached to a side surface of the optical fiber 3 extending through the fiber hole 5a. A central axis 5b of the fiber hole 5a is aligned with a central axis of the main unit 5, and the optical fiber 3 is disposed coaxially with the main unit 5 (see FIG. 3A and FIG. 3B). A cantilevered distal end portion 3b protruding from the distal end surface of the main unit 5 of the optical fiber 3 is a vibration region that vibrates in the radial direction. The vibration of the vibration region 3b causes the laser light L output from the distal end 3a to be scanned.

In order to obtain a larger amplitude of the vibration region 3b within the channel 2b, the central axis 5b is preferably aligned with the central axis of the main unit 5. However, the central axis 5b does not necessarily have to be aligned with the central axis of the main unit 5. For example, the central axis 5b may be decentered from the central axis of the main unit 5 within a range in which a required amplitude of the vibration region 3b can be obtained within the channel 2b. For example, the central axis 5b may be decentered from the central axis of the main unit 5 by Several hundred μm.

The main unit 5 and the retainer 1 each have an outer diameter slightly smaller than the inner diameter of the channel 2b. The main unit 5 is disposed in an annular space between the inner surface of the channel 2b and the side surface of the optical fiber 3, whereby a substantially fixed distance is maintained between the side surface of the optical fiber 3 and the inner surface of the channel 2b. Specifically, the position of a part of the optical fiber 3 in the main unit 5 is substantially fixed in the radial direction within the channel 2b, and the vibration region 3b vibrates about the part of the optical fiber 3 in the main unit 5 acting as a fulcrum P.

In one design example, the inner diameter of the channel 2b is 1.2 mm, the outer diameter of the optical fiber 3 ranges between 0.15 mm and 0.20 mm, and the outer diameter of the main unit 5 is 1.1 mm.

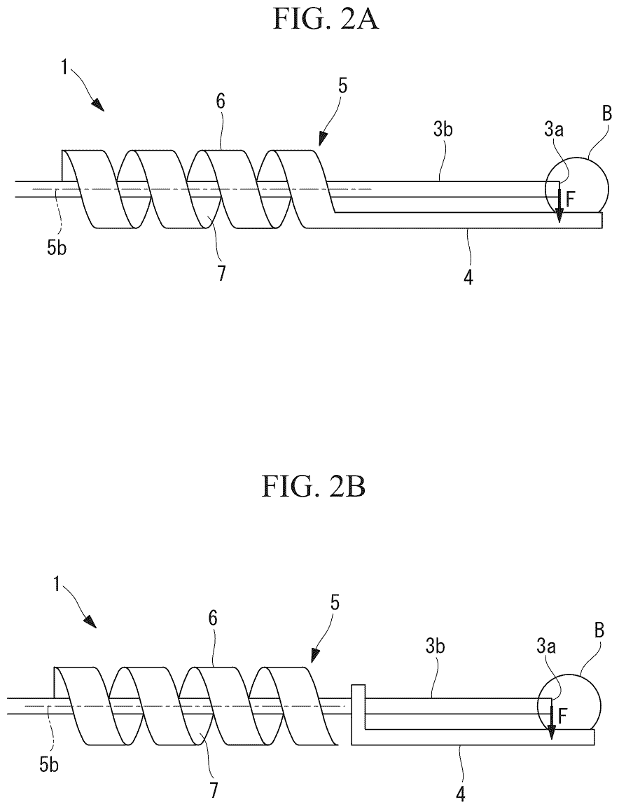

As shown in FIG. 2A, the main unit 5 has a helical structural body 6 wound around the central axis 5b and disposed around the fiber hole 5a, and also has a flow-path defining section 7 that defines, within the channel 2b, a flow path through which the perfusate D can travel in the longitudinal direction.

As shown in FIG. 3A, the structural body 6 has a rib-like helical end surface 6a that protrudes radially inward and that defines the fiber hole 5a at the radially inner side. The structural body 6 is in contact with the side surface of the optical fiber 3 at the end surface 6a to support the optical fiber 3.

The structural body 6 is composed of an elastic material, and the diameter of the fiber hole 5a is smaller than the outer diameter of the optical fiber 3. As shown in FIG. 3B, the optical fiber 3 is inserted into the fiber hole 5a while elastically deforming the rib of the structural body 6. The elastically-deformed rib squeezes the optical fiber 3, so that the main unit 5 becomes securely fixed to the side surface of the optical fiber 3. By pulling out the optical fiber 3 from the fiber hole 5a, the retainer 1 can be detached from the optical fiber 3.

The helical structural body 6 is wound with a gap in-between in the longitudinal direction. The gap forms a helical recess that continuously extends from the proximal end surface to the distal end surface of the structural body 6 and that functions as a flow path for the perfusate D. The flow-path defining section 7 is constituted of the helical recess. As shown in FIG. 3C, in a cross section of the main unit 5 at each position in the longitudinal direction, the structural body 6 is disposed in one area in the circumferential direction, and the recess 7 is formed in another area in the circumferential direction where the structural body 6 is not disposed. Accordingly, the recess 7 has a large cross-sectional area and allows for a high perfusion flow rate (e.g., 20 ml/minute) while enabling the main unit 5 to have a small diameter.

As shown in FIG. 3A, a proximal end surface 5c of the main unit 5 (i.e., the proximal end surface of the structural body 6) is preferably a slope that is inclined radially inward toward the distal end. When the distal end 3a of the optical fiber 3 is inserted into the fiber hole 5a from the proximal end surface 5c, the distal end 3a is guided toward the center in accordance with the inclination of the proximal end surface 5c, so as to be automatically positioned in the fiber hole 5a. This facilitates the insertion of the optical fiber 3 into the fiber hole 5a and the attachment of the retainer 1 to the optical fiber 3.

In FIG. 3A, in addition to the proximal end surface 5c, a helical surface oriented toward the proximal end of the structural body 6 is also inclined over the entire length, similarly to the proximal end surface 5c.

The number of turns of the helical structural body 6 is preferably two or more. The structural body 6 whose number of turns is two or more supports the side surface of the optical fiber 3 at three or more locations in a vertical section taken at any angle around the central axis 5b, whereby the fulcrum P can be made more positionally stable.

The structural body 6 in FIG. 3A has a fixed pitch α and a fixed width β.

As shown in FIG. 4, the pitch α and the width β may vary depending on the position in the longitudinal direction. A retention force at each position of the main unit 5 relative to the optical fiber 3 varies depending on the width β at the position. The width β at each position may be designed in accordance with the required retention force.

As shown in FIG. 2A, the operation member 4 is a vibration mechanism that vibrates the distal end 3a of the optical fiber 3 in the radial direction by utilizing a suction effect of a bubble B. A suction effect is a phenomenon where the distal end 3a is drawn toward the operation member 4 due to contraction of the bubble B.

The pulsed laser light L output from the distal end 3a increases the temperature of a liquid medium surrounding the distal end 3a, thereby generating the bubble B at the distal end 3a. The bubble B repeatedly undergoes generation, growth, contraction, and collapse in synchronization with the pulsed laser light L.

As shown in FIG. 2A, the operation member 4 is a plate-shaped member disposed parallel to the vibration region 3b. The operation member 4 is disposed only at one side of the distal end 3a in the radial direction, and is disposed at a position where the bubble B can come into contact therewith from the distal end 3a with a distance therebetween in the radial direction. The operation member 4 causes a contraction force F to act on the distal end 3a toward the operation member 4 during contraction of the bubble B, whereby the vibration region 3b vibrates.

The operation member 4 is integrated with the main unit 5 and serves as a part of the retainer 1. Specifically, the retainer 1 includes the operation member 4 in addition to the main unit 5. For example, the operation member 4 protrudes in the longitudinal direction from one circumferential area of the distal end surface of the main unit 5.

Since the insertion section 2a bends within the body C, the retainer 1 and the operation member 4 are preferably flexible enough to be extendable through the channel 2b that is bent. Moreover, the retainer 1 and the operation member 4 preferably have elasticity that allows them to recover their original shapes after being removed from the channel 2b. For example, the retainer 1 and the operation member 4 are composed of flexible resin.

The main unit 5 constituted of the helical structural body 6 has high flexibility against bending. Therefore, while flexibly bending in accordance with the bending of the channel 2b, the retainer 1 can smoothly extend through the bent channel 2b.

The laser light source 20 is, for example, a laser oscillator and is optically connected to the proximal end of the optical fiber 3. In response to an operation performed on a foot switch 20a, the laser light source 20 outputs the pulsed laser light L for treating the target A. The laser light L is, for example, infrared light.

The fluid supply source 30 is fluidically connected to the proximal end of the channel 2b and supplies the perfusate D, such as a physiological saline solution, to the channel 2b. For example, the fluid supply source 30 includes a container for holding the perfusate D and a pump for sending the perfusate D from the container to the channel 2b.

The controller 40 controls the pulse frequency of the laser light L output by the laser light source 20. For example, the controller 40 controls the pulse frequency of the laser light L to a frequency equal to the resonance frequency of the vibration region 3b.

Next, a phototherapy method using the phototherapy device 10 and the phototherapy system 100 will be described.

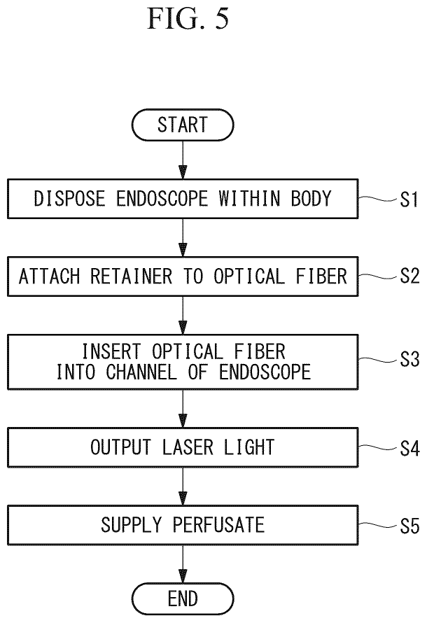

As shown in FIG. 5, the phototherapy method includes step S1 for disposing the endoscope 2 within the body C, step S2 for preparing the optical fiber 3 having the retainer 1 attached thereto, step S3 for inserting the optical fiber 3 having the retainer 1 attached thereto into the channel 2b of the endoscope 2, step S4 for irradiating the target A with the laser light L, and step S5 for supplying the perfusate D.

An operator, such as a surgeon, inserts the endoscope 2 into, for example, a kidney through the urethra (step S1).

Then, the operator inserts the optical fiber 3 into the fiber hole 5a via the proximal end surface 5c, thereby attaching the main unit 5 to the side surface of the optical fiber 3 and attaching the operation member 4 to the optical fiber 3 via the main unit 5 (step S2). Alternatively, the optical fiber 3 having the main unit 5 and the operation member 4 preliminarily attached thereto may be provided to the operator.

Subsequently, the operator inserts the optical fiber 3 having the main unit 5 and the operation member 4 attached thereto into the channel 2b, disposes the distal end 3a of the optical fiber 3 outside the channel 2b, and disposes the main unit 5 inside the channel 2b (step S3).

Then, the operator steps on the foot switch 20a to cause the laser light source 20 to start outputting the laser light L (step S4). The pulsed laser light L is radiated onto the target A from the distal end 3a of the optical fiber 3, thereby fragmenting a calculus serving as the target A.

In this case, the pulsed laser light L is repeatedly output from the distal end 3a, so that the bubble B is repeatedly generated and collapsed at the distal end 3a, whereby the operation member 4 causes the force F to act on the distal end 3a every time the bubble B contracts. Consequently, the vibration region 3b of the optical fiber 3 vibrates about the position of the main unit 5 within the channel 2b acting as the fulcrum P, whereby the laser light L is scanned over the target A.

Step S5 is performed concurrently with step S4 and involves the operator supplying the perfusate D to the channel 2b from the fluid supply source 30 (step S5). The perfusate D travels through the helical recess 7 of the retainer 1 within the channel 2b, and is discharged from a distal-end opening of the channel 2b. The discharged perfusate D enables improvement of poor visibility of the endoscope 2 caused by fragments of the calculus, whereby a clear visual field is obtained.

Accordingly, in this embodiment, the retainer 1 attached to the optical fiber 3 allows the fulcrum P to be fixed within the channel 2b. Consequently, the vibration region 3b vibrates stably, and a large vibration amplitude of the distal end 3a is obtained. As a result, the laser light L can be scanner over a wide range of the target A, thereby achieving enhanced treatment efficiency, such as enhanced calculus fragmentation efficiency, using the laser light L.

During calculus treatment, a large fragmentation amount of the calculus A causes a large number of fragments to occur. In this embodiment, in a state where the main unit 5 is disposed within the channel 2b, the helical recess 7 provided in the main unit 5 defines, within the channel 2b, a flow path through which the perfusate D can travel in the longitudinal direction. Thus, the perfusate D can be supplied to the visual field of the endoscope 2 via the channel 2b without being blocked by the retainer 1, so that the target A can be treated using the laser light L while a clear visual field is maintained by the perfusate D. Accordingly, the fixation of the fulcrum P and the perfusion can both be achieved by using the single channel 2b having a small diameter.

In particular, the main unit 5 constituted of the helical structural body 6 can ensure a large cross-sectional area of the flow path within the channel 2b having the small diameter, so that the perfusate can flow therethrough at a high flow rate. Moreover, as shown in FIG. 3B, in any vertical section, the helical structural body 6 alternately comes into contact with opposite sides of the side surface of the optical fiber 3. Accordingly, the structural body 6 can stably retain the optical fiber 3 and can stably fix the position of the fulcrum P.

Furthermore, in this embodiment, the optical fiber 3 that is coaxial with the main unit 5 is disposed on the central axis of the channel 2b. Thus, the inner diameter of the channel 2b can be maximally utilized to vibrate the distal end 3a of the optical fiber 3 with a large amplitude.

Supposing that the optical fiber 3 is decentered relative to the central axis of the channel 2b, interference of the vibrating optical fiber 3 with the inner surface of the channel 2b may make it difficult to increase the amplitude of the optical fiber 3.

The retainer 1 can be readily attached to and detached from the optical fiber 3 by simply inserting and removing the optical fiber 3 into and from the fiber hole 5a. Therefore, the retainer 1 or the optical fiber 3 can be readily replaced, where necessary, during treatment of the calculus A.

As an alternative to the above embodiment in which the structural body 6 is helical, the structural body 6 may have a different structure. In detail, the structural body may have a structure in which it is disposed in one area in the circumferential direction at each position in the longitudinal direction such that another area in the circumferential direction forms a recess extending continuously from the proximal end to the distal end of the structural body. For example, the structural body may have multiple ribs that are arranged with a distance therebetween in the circumferential direction that extend parallel to the central axis 5b, and may have a recess between the ribs.

As an alternative to the above embodiment in which the operation member 4 is integrated with the main unit 5, the operation member 4 may be separated from the main unit 5, as shown in FIG. 2B. For example, the proximal end of the operation member 4 may be fixed to the side surface of the optical fiber 3.

Accordingly, when the operation member 4 is independent of the retainer 1, the retainer 1 and the operation member 4 are independently attached to the optical fiber 3 in step S2.

Although the flow-path defining section 7 is a helical recess provided in the main unit 5 in the above embodiment, the flow-path defining section 7 is not limited to this type and may be of a different type so long as the perfusate D can travel in the longitudinal direction in the region of the retainer 1 within the channel 2b.

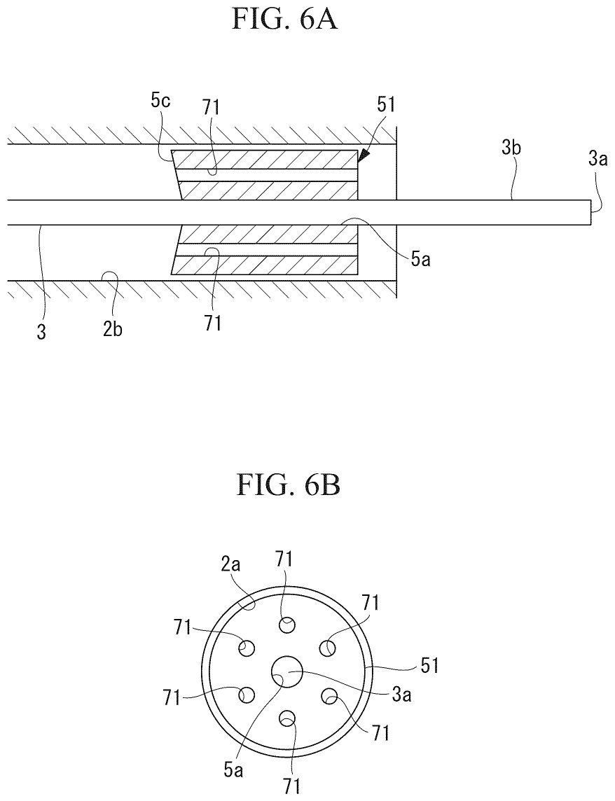

FIG. 6A to FIG. 8 each illustrate another configuration example of the main unit 5. Any of main units 51, 52, and 53 in FIG. 6A to FIG. 8 can fix the fulcrum P, can ensure the flow path within the channel 2b, and can be readily attached to and detached from the optical fiber 3. In FIG. 6A to FIG. 8, the operation member 4 is not shown.

In FIG. 6A and FIG. 6B, the main unit 51 is constituted of a cylindrical body, and the flow-path defining section is constituted of one or more through-holes 71 provided in the main unit 51. Each through-hole 71 extends through the main unit 51 in the longitudinal direction from the proximal end surface to the distal end surface of the main unit 51, and forms a flow path. In order to prevent the optical fiber 3 from being erroneously inserted into the through-holes 71, each through-hole 71 preferably has a dimension smaller than the outer diameter of the optical fiber 3 in at least one radial direction.

In the example in FIG. 6A and FIG. 6B, each through-hole 71 is a circular hole having a diameter smaller than the outer diameter of the optical fiber 3. Each through-hole 71 may have a shape other than a circular shape. By increasing the number of through-holes 71 or the cross-sectional area of each through-hole 71, the overall cross-sectional area of the flow path for the perfusate D can be increased.

In FIG. 7, the main unit 52 has a two-step structure including a distal end 52a and a proximal end 52b each having through-holes 71. The distal end 52a and the proximal end 52b are connected to each other by a thin-walled connector 52c, and have a space between the distal end 52a and the proximal end 52b.

With this main unit 52, high flexibility against bending can be achieved.

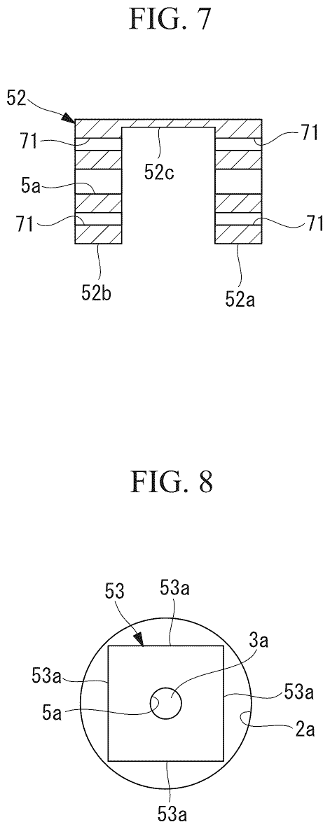

In FIG. 8, the main unit 53 is a tubular body having a cross section with an outer shape different from the cross-sectional shape of the channel 2b, and has side surfaces 53a disposed at positions located away from the inner surface of the channel 2b in the radial direction of the channel 2b. The flow-path defining section is constituted of the side surfaces 53a, and defines the flow path between the inner surface of the channel 2b and the side surfaces 53a.

In the example in FIG. 8, the channel 2b has a circular cross section, and the main unit 53 has a rectangular tubular shape with four side surfaces 53a. The main unit 53 may have another polygonal tubular shape or an elliptical cylindrical shape. If the channel 2b has a non-circular cross section, the main unit 53 may be cylindrical.

By utilizing the space between the side surfaces 53a of the main unit 53 and the inner surface of the channel 2b in this manner, a flow path with a large cross-sectional area can be defined within the channel 2b while the main unit 53 has a simple structure.

Although a medical tube serves as the endoscope 2 in the above embodiment, the medical tube may be any medical device that has a channel, and may be, for example, a catheter.

As an alternative to the above embodiment in which the retainer 1 is detachably fixed to the side surface of the optical fiber 3, the retainer 1 may be fixed to the side surface of the optical fiber 3 in a non-detachable manner. For example, the inner surface of the fiber hole 5a may be fixed to the side surface of the optical fiber 3 by using an adhesive.

As an alternative to the above embodiment in which the retainer 1 is attached to the optical fiber 3, the retainer 1 may be provided at the inner surface of the channel 2b in the vicinity of the distal end of the channel 2b and may be integrated with the channel 2b.

In this case, the optical fiber 3 alone is inserted into the channel 2b in step S3, and the optical fiber 3 extends through the fiber hole 5a so that the main unit 5 retains the optical fiber 3.

As an alternative to the above embodiment in which the phototherapy device 10 includes the operation member 4 as a vibration mechanism, the phototherapy device 10 may include a different vibration mechanism. For example, the vibration mechanism may be a piezoelectric actuator using a piezoelectric element or may be an electromagnetic actuator using a permanent magnet or an electromagnetic coil.

Although the embodiment of the present invention and the modifications thereof have been described above, the present invention is not limited thereto and are modifiable, as appropriate, within a range not departing from the scope of the present invention.

For example, the main unit of the optical fiber retainer may have two or more flow-path defining sections among the recess 7, the through-holes 71, and the side surfaces 53a.

The phototherapy device 10 and the phototherapy system 100 are not limited to lithotripsy and are applicable to any treatment involving irradiating a target with light. In particular, the phototherapy device 10 and the phototherapy system 100 may be suitably applied to treatment performed while supplying a liquid or gas. The fluid supplied to the channel 2b by the fluid supply source 30 is also appropriately selected depending on the type of treatment. Specifically, the fluid supply source 30 may supply a different liquid or gas to the channel 2b.

### REFERENCE SIGNS LIST

1 optical fiber retainer
2 endoscope (medical tube)
2b channel
3 optical fiber
3a distal end
4 operation member (vibration mechanism)
5, 51, 52, 53 main unit
53a side surface (flow-path defining section)
5a fiber hole
5b central axis
5c proximal end surface
6 structural body
7 recess (flow-path defining section)
71 through-hole (flow-path defining section)
10 phototherapy device
20 laser light source
30 fluid supply source
100 phototherapy system

1. An optical fiber retainer that is attached to a side surface of an optical fiber and that maintains, within a channel of a medical tube, a fixed distance between the side surface of the optical fiber and an inner surface of the channel, the optical fiber retainer comprising:
a substantially tubular main unit having a fiber hole through which the optical fiber extends,
wherein the main unit has a flow-path defining section that defines a flow path within the channel, the flow path allowing a fluid to travel in a longitudinal direction of the main unit.

2. The optical fiber retainer according to claim 1,
wherein the main unit has a rib-shaped structural body that is disposed around the fiber hole and that is in contact with the side surface of the optical fiber to support the optical fiber,
wherein the structural body is disposed in one area in a circumferential direction at each position in the longitudinal direction, and another area in the circumferential direction forms a recess extending continuously from a proximal end to a distal end of the structural body, and
wherein the flow-path defining section is constituted of the recess.

3. The optical fiber retainer according to claim 2,
wherein the structural body has a helical shape wound around a central axis of the fiber hole with a gap in-between in the longitudinal direction, and
wherein the gap forms the recess having a helical shape.

4. The optical fiber retainer according to claim 1,
wherein the main unit is constituted of a tubular body, and
wherein the flow-path defining section is constituted of at least one through-hole that extends through the main unit in the longitudinal direction and that forms the flow path.

5. The optical fiber retainer according to claim 1,
wherein the main unit is constituted of a tubular body having a cross section with an outer shape different from a cross-sectional shape of the channel, and
wherein the flow-path defining section is constituted of a side surface of the main unit disposed at a position located away from the inner surface of the channel.

6. The optical fiber retainer according to claim 5, wherein the main unit has a cylindrical shape, an elliptical cylindrical shape, or a polygonal tubular shape.

7. The optical fiber retainer according to claim 1, wherein a proximal end surface of the main unit is a slope that is inclined toward a distal end and radially inward in a direction intersecting the longitudinal direction.

8. The optical fiber retainer according to claim 1, further comprising an operation member that protrudes in the longitudinal direction of the main unit from a distal end surface of the main unit and that is disposed only at one side of a distal end of the optical fiber in a radial direction intersecting the longitudinal direction.

9. A phototherapy device comprising:
a medical tube having a channel;
an optical fiber inserted in the channel; and
an optical fiber retainer that is disposed between the optical fiber and an inner surface of the channel and that maintains a fixed distance between a side surface of the optical fiber and the inner surface of the channel,
wherein the optical fiber retainer includes a substantially tubular main unit having a fiber hole through which the optical fiber extends, and
wherein the main unit has a flow-path defining section that defines a flow path within the channel, the flow path allowing a fluid to travel in a longitudinal direction of the main unit.

10. The phototherapy device according to claim 9, wherein the medical tube is an endoscope.

11. The phototherapy device according to claim 9, wherein the optical fiber retainer is attached to the side surface of the optical fiber or is provided at the inner surface of the channel.

12. The phototherapy device according to claim 9, further comprising a vibration mechanism configured to vibrate a distal end of the optical fiber in a radial direction of the optical fiber.

13. The phototherapy device according to claim 12, wherein the vibration mechanism includes an operation member disposed only at one side of the distal end of the optical fiber in the radial direction of the optical fiber.

14. A phototherapy system comprising:
a medical tube having a channel;
an optical fiber inserted in the channel;
an optical fiber retainer that is disposed between the optical fiber and an inner surface of the channel and that maintains a fixed distance between a side surface of the optical fiber and the inner surface of the channel;
a laser light source that supplies laser light to the optical fiber; and
a fluid supply source that supplies a fluid to the channel,
wherein the optical fiber retainer includes a substantially tubular main unit having a fiber hole through which the optical fiber extends, and
wherein the main unit has a flow-path defining section that defines a flow path within the channel, the flow path allowing the fluid to travel in a longitudinal direction of the main unit.
